(19) United States
(12) Patent Application Publication
(10) Pub. No.: US 2010/0036471 A1
(43) Pub. Date: Feb. 11, 2010

(54) Method of Diagnosing and Treating Lower Urinary Tract Symptoms

(75) Inventors: Mark J. Dolan, Santa Rosa, CA (US); Dwayne S. Yamasaki, St. Augustine, FL (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/186,360

(22) Filed: Aug. 5, 2008

(51) Int. Cl. A61F 2/84; A61B 5/05

(52) U.S. Cl. 623/1.11; 600/407

(57) ABSTRACT

A method of diagnosing and treating a patient having lower urinary tract symptoms caused by insufficient blood flow to the urinary bladder, the urethra, a nerve innervating the urinary bladder, or a nerve innervating the urethra due to atherosclerosis of a pelvic artery is disclosed. A method of diagnosing the patient's condition includes determining if a stenosis exists within a pelvic vessel. A method of treating the patient's condition may include placing a stent within the stenosed pelvic artery.

### FIELD OF THE INVENTION

The present invention relates to a method of diagnosing and treating lower urinary tract symptoms. More particularly, a method of diagnosing a patient having lower urinary tract symptoms caused by atherosclerosis into or through a pelvic artery for treatment by placement of a stent within the stenosed region of the affected vessels.

### BACKGROUND OF THE INVENTION

Lower Urinary Tract Symptoms (LUTS) is a common problem for both men and women that adversely affects the health and quality of life of millions of people. Individuals with urinary control disorders often face debilitating challenges in their everyday lives. These individuals may be preoccupied with trips to the bathroom, fears of embarrassment and sleepless nights. Some sufferers become so anxious that they become isolated and depressed. Literature supports LUTS etiology as multifactorial, common factors being age, childbirth, fecal difficulties, obstetric complications, obesity, pelvic surgery, medications, functional impairment, chronic diseases, menstrual cycle, race, family history, menopause, and Benign Prostatic Hyperplasia or BPH.

Symptoms of LUTS include urinary incontinence, or the inability to control the passage of urine. Although there are a variety of different types of urinary incontinence, stress incontinence, urge incontinence and urinary retention are the most common. Stress incontinence is the unacceptable passage of urine under the stress of increased abdominal pressure. This increased pressure typically results from coughing, sneezing, and Valsalva. This disorder is most common in multiparous, post-menopausal females. Urge incontinence is the involuntary passage of urine with a concomitant sense of urgency. Systometry indicates detrusor (urinary bladder wall muscle) contractions with low urinary bladder filling pressures and volumes. Unsolicited, premature bladder contraction may result from mucosal irritation of varied etiology. Urinary retention is characterized by the inability of a patient to spontaneously and controllably urinate. Catheterization of the urethra is provided to many patients suffering from urinary retention, which is often a painful and somewhat lengthy procedure having the added risk of causing infection. Symptoms of LUTS also include difficult or painful urination (dysuria), excessive urination at night (nocturia), poor stream, hesitancy, terminal dribbling, incomplete voiding, and overflow incontinence.

Treatments for LUTS range from observation to medical treatment to surgery. More specifically, LUTS may be treated with a variety of therapeutic options such as pharmacological treatment, mechanical intervention such as self-catheterization of the urethra, physical appliances such as diapers, and surgical intervention. Surgical treatments are the most invasive and are often considered after other therapies have proven ineffective. In certain instances, the implantation of a prostatic stent within the narrowed area of the urethra may be medically necessary due to the extent of the urethral blockage and/or if the age or health of the patient makes him a poor surgical candidate. Placement of a temporary prostatic stent as a differential diagnosis test can help identify whether LUTS symptoms are directly related to obstruction of the prostate. In such a procedure, the prostatic stent is tracked through the urethra to the narrowed or obstructed area and allowed to expand, to push back the prostatic tissue and widen the urethra. However the American Urological Association cautions that the placement of prostatic stents should be considered only in high-risk patients, for example, those with urinary retention, because prostatic stents are associated with significant complications, such as encrustation, infection and chronic pain.

Accordingly, current treatments for LUTS may be invasive and/or have unpleasant side effects. In addition, although there are many treatments and therapies for LUTS, some cases of LUTS may not be correctable or may be better addressed by a therapy other than those presently available. Accordingly, what is needed is a method of diagnosing and treating lower urinary tract symptoms of an etiology not addressed by existing therapies.

### BRIEF SUMMARY OF THE INVENTION

An embodiment of the present invention is a method of diagnosing lower urinary tract symptoms that is caused by insufficient blood flow to the urinary bladder, urethra, nerves that innervate the urinary bladder, or nerves that innervate the urethra due to atherosclerosis of an artery that supplies blood to those structures. In almost all cases, the artery is a pelvic artery, but there may be instances of other arteries that feed the bladder due to variations in the vascular anatomy. In a further embodiment, the artery is a superior vesical artery, an inferior vesical artery, an umbilical artery, and/or a vaginal artery. Diagnosing a stenosis in a pelvic artery requires imaging of the target arteries by performing a diagnostic procedure such as angiography, a CT scan, a MRI, CT-angiography, MR-angiography, doppler sonography, color duplex sonography, or nuclear imaging.

Another embodiment of the present invention is a method of treating lower urinary tract symptoms caused by atherosclerosis in a pelvic artery. The method includes tracking a stent delivery catheter to a stenosis within the pelvic artery and delivering a stent within the stenosis to restore flow through the pelvic artery. In an embodiment, the pelvic artery is one of the superior vesical artery, the inferior vesical artery, the umbilical artery, the vaginal artery, the internal pudendal, the obturator, and the middle rectal artery that is reached by tracking the stent delivery catheter through the femoral artery, the internal iliac artery and then into one of the superior vesical artery, the inferior vesical artery, the umbilical artery, the vaginal artery, the internal pudendal, and the middle rectal artery.

In various other embodiments, the revascularization of the pelvic artery in accordance with embodiments of the present invention may include angioplasty, rotational atherectomy, ultrasonic or other vibrational mechanisms to break-up the stenosis with or without aspiration, intravascular brachytherapy, stenotic maceration such as by angiojets, clot retrieval, and/or drug/biologic delivery.

### BRIEF DESCRIPTION OF DRAWINGS

The foregoing and other features and advantages of the invention will be apparent from the following description of the invention as illustrated in the accompanying drawing. The accompanying drawing, which is incorporated herein and forms a part of the specification, further serves to explain the principles of the invention and to enable a person skilled in the pertinent art to make and use the invention. The drawing is not to scale.

FIG. 1 is an illustration of a guide catheter and stent delivery system for use in a method of treating lower urinary tract symptoms according to an embodiment of the present invention.

### DETAILED DESCRIPTION OF EMBODIMENTS OF THE INVENTION

The following detailed description is merely exemplary in nature and is not intended to limit the invention or the application and uses of the invention. Furthermore, there is no intention to be bound by any expressed or implied theory presented in the preceding technical field, background, brief summary or the following detailed description.

Lower urinary tract symptoms have several etiologies, one of which may be insufficient blood flow through the pelvic arteries that feed the urinary bladder, urethra, nerves that innervate the urinary bladder, or nerves that innervate the urethra. The urinary bladder is a hollow, muscular, and distensible organ that sits on the pelvic floor in mammals. It is the organ that collects urine excreted by the kidneys prior to disposal by urination. Atherosclerosis occurring anywhere along the arterial path that supplies blood to the bladder, the urethra, or the nerves innervating the bladder or urethra may result in insufficient blood supply to various parts of those structures, resulting in poor muscular contraction and/or poor sensing of bladder distension, which in turn may cause LUTS. As such, stenting or another revascularization procedure performed within the pelvic arterial region may be effective in treating lower urinary tract symptoms caused by the vascular disease by increasing blood flow to the bladder or urethra.

Insufficient blood flow may occur through an artery that feeds the urinary bladder or urethra. The blood supply of the bladder is derived from the superior and inferior vesical arteries, the umbilical artery, and in females, the vaginal artery. The obturator and inferior gluteal arteries also give off branches to the bladder. The superior vesical, inferior vesical, umbilical, vaginal, obturator, and inferior gluteal arteries are branches of the internal iliac artery, also called the hypogastric artery, which is a branch of the common iliac arteries. The internal iliac artery is the main artery of the pelvis. The arteries feeding the male urethra include the prostatic arteries, artery to the urethral bulb, and urethral artery. Blood feeding the female urethra is from the inferior vesical, internal pudendal, and vaginal arteries. In addition, insufficient blood flow may also occur through a pelvic artery that feeds nerves that innervate the urinary bladder or the urethra. The innervation to the bladder is from the vesical nerve plexus (parasympathetic pelvic splanchnic nerves, and from sympathetic fibers from T11-L2). Innervation of the urethra is via branches of the pudendal nerve (deep perineal nerve), and autonomic innervation is from the cavernous nerve plexuses (in the male). Atherosclerosis occurring in an artery that supplies one of the above-mentioned nerves anywhere along its entire length may result in insufficient blood flow and/or impaired functionality of the bladder or the urethra.

In addition, insufficient blood flow through the pelvic arteries that feed the prostate may result in Benign Prostatic Hyperplasia or BPH, another etiology of LUTS. The prostate is a walnut sized gland found beneath the bladder and in front of the rectum that surrounds part of the male urethra. BPH refers to a condition in which an enlarged prostate presses against the urethra and interferes with urination, resulting in LUTS. The arteries of the prostate are derived from the internal pudendal, inferior vesicle, and middle rectal arteries. The internal pudendal, inferior vesical, and/or middle rectal arteries are also branches of the internal iliac artery.

In accordance with an embodiment of the present invention, a patient first undergoes a diagnostic procedure in order to determine whether a revascularization procedure is likely to alleviate lower urinary tract symptoms in the patient. An initial step in making the diagnosis is to perform a pelvic angiogram on the patient. An angiogram is a diagnostic procedure using an x-ray to visualize blood vessels by following the introduction of a contrast material through an artery or system of vessels. A pelvic angiogram showing a stenosis in one of the pelvic arteries, such as the superior vesical, the inferior vesical, the umbilical, the vaginal, the internal pudendal, or middle rectal arteries, may be treatable with a stent or other revascularization procedure to increase blood flow to the bladder and/or prostate arteries. In another embodiment of the present invention, other diagnostic procedures, such as a CT scan, MRI, CT-angiography (CTA), MR-angiography (MRA), indirectly via doppler or color duplex sonography, and nuclear imaging, i.e., radionuclide angiography, may be used to determine whether a stenosis exists in one of the pelvic arteries.

Upon diagnosis of a stentable or otherwise treatable stenosis within one of the patient's pelvic arteries, such as the superior or inferior vesical arteries, treatment of the patient's lower urinary tract symptoms may be effectuated by placement of a stent. In accordance with various embodiments of the present invention, the stent may be one of a self-expanding stent or a balloon expandable stent that is placed within the stenosed region of the pelvic vasculature. The restoration of blood flow to the ischemic region improves bladder function either acutely or chronically.

Due to the position of the arteries of the pelvis within a person's anatomy, it may receive in vivo loading from forces external to the body that may potentially crush the artery, as well as any implanted stent therein, between the external force and the sacrospinous ligament. As such, if the stenosis to be treated is within a pelvic artery, a self-expanding stent design may be warranted so that if subjected to acute high forces within the artery the self-expanding stent design will deform and then return to its original configuration. Thus in an embodiment of the present invention, a self-expanding stent may be implanted within the superior or inferior vesical artery to provide vessel support within a stenosed region of the artery. The stent may be delivered to the treatment site by tracking a stent delivery catheter through an access site in the femoral artery then into the internal iliac artery and subsequently into the superior or inferior vesical artery.

In addition, though contrary to conventional wisdom, recent testing performed by the inventors on a series of cadavers has proven that a balloon expandable stent design with sinusoidal elements was able to withstand direct external loading at the sacrospinous ligament and perineal loading directed at a balloon expanded stent implanted within the internal pudendal artery. Current balloon expanded stent designs would be desirable for use within the internal pudendal artery as, unlike self-expanding stents, they are readily available with outer expanded diameters that are small enough yet strong enough to be supportively implanted within the extremely narrow distal regions of the internal pudendal artery. Thus in another embodiment of the present invention, a balloon expandable stent may be implanted within a target pelvic artery to provide vessel support within a stenosed region of the artery. The stent may be delivered to the treatment site by tracking a stent delivery catheter through an access site in the femoral artery then into the internal iliac artery and subsequently into the target pelvic artery.

In various other embodiments of diagnosis and treatment of a pelvic artery in accordance with the present invention, the pelvic artery may be one of the common iliac artery, external iliac artery, and/or internal iliac or hypogastric artery. In addition to stenting, embodiments of the present invention include other procedures for revascularization of the pelvic artery. For instance, angioplasty, also known as balloon dilation or balloon angioplasty, is an effective therapy for some patients with artery disease and may be used to dilate or widen narrowed arteries. Balloon angioplasty utilizes a catheter with a deflated balloon on its tip for positioning within the narrowed part of the pelvic artery. Once properly positioned, the balloon is inflated and the narrowed area of the pelvic artery is widened to restore blood flow therethrough.

In another embodiment, a rotational atherectomy or rotoblading procedure may be preferred if the pelvic artery is blocked or occluded by a hard plaque. A rotational atherectomy may utilize a catheter with a high-speed burr, such as one coated with diamond tips, to grind the hard plaque inside the pelvic artery into fine particles that then pass downstream without blocking the patient's circulation. Alternatively, a distal protection device, such as a filter may be positioned downstream of the treatment site to trap embolic debris for subsequent removal.

In another embodiment where the stenosis is caused by a thromboembolic event, revascularization may include softening or breaking-up the stenosis or clot and then aspirating the particulate from the pelvic artery. The stenosis, whether a clot or obstruction, may be initially treated, i.e., loosened and/or broken up, through the use of an ultrasonic medical device, such as any of the devices disclosed in U.S. Pat. No. 6,660,013 to Rabiner et al. and U.S. Pat. No. 6,652,547 to Rabiner et al., each of which is assigned to OmniSonics Medical Technologies, Inc. of Wilmington, Mass., or by another mechanical disruption provided by, for e.g., sinusoidal wires, coils and the like, such as those available from EKOS Corporation of Bothell, Wash. A method in accordance with an embodiment of the present invention, includes tracking an ultrasonic guidewire or catheter to the obstruction within the lumen of an aspiration catheter and then activating the guidewire to break up the obstruction while the debris is aspirated through a catheter. Alternatively, initial treatment may include stenotic maceration by venturi and saline jet devices, such that the stenosis is adequately softened or thinned to be removed by a clot retrieval device or by aspiration.

In another embodiment, revascularization of the pelvic artery may include intravascular brachytherapy utilizing a catheter for delivering a radioactive source to the stenosis, such that the radioactive source may be placed within or proximate the vascular stenosis for radiation treatment.

In another embodiment, a drug-eluting stent or drug-eluting implant (hydrogel, endoluminal paving, polymeric tube, etc) can be implanted in the arteries feeding the bladder for local delivery of drugs to the bladder. Local drug delivery has the advantage of maximizing the dose to the target organ, while minimizing its effect systemically. In one embodiment, the stent or implant may be coated with a therapeutic substance to provide short term therapeutic value. In another embodiment, the stent or implant may be coated with a therapeutic substance to prevent restenosis, such as a therapeutic substance from the limus drug family or paclitaxol. Other suitable examples include, but are not limited to, antiplatelet agents, anticoagulant agents, antimicrobial agents, antiinflammatory, and antimetabolic agents.

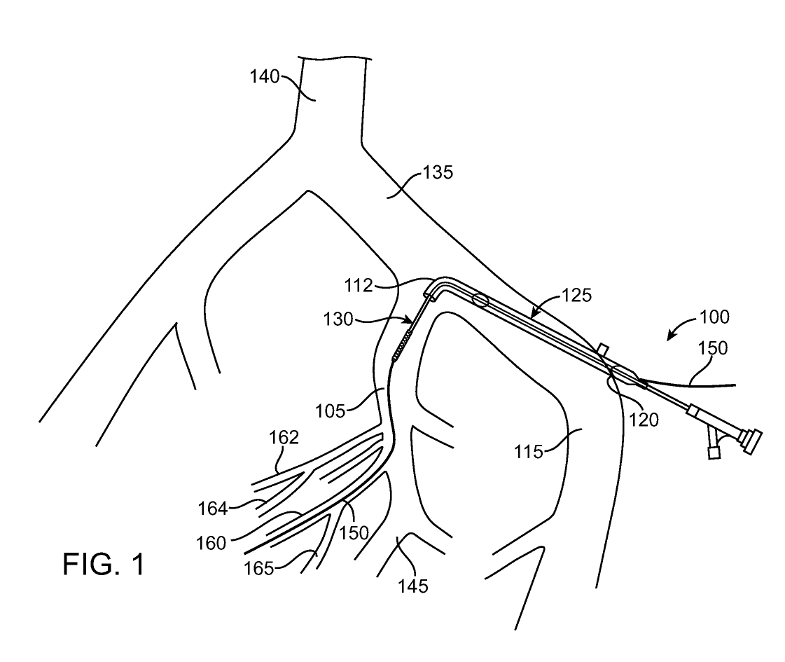

As shown in FIG. 1, a guide catheter and stent delivery system 100 may be used in embodiments of the present invention to more easily navigate the pelvic vasculature. System 100 is more fully disclosed in U.S. Patent Appl. Pub. No. US2006/0079951 A1 to Dolan et al., which is incorporated by reference herein in its entirety. System 100 is introduced into femoral artery 115 through a percutaneous access site 120 to be tracked over a guidewire 150, which in FIG. 1, extends within the pelvic vasculature to the treatment site (not shown) within the inferior vesical artery 160. System 100 includes a guide catheter portion 125 shown passing within femoral artery 115 and having an angled tip 112 directed into an ostium of internal iliac artery 105. A stent delivery portion 130 of system 100 is shown extending into internal iliac artery 105 from guide catheter portion 125. Stent delivery portion 130 is then maneuverable through internal iliac artery 105 into inferior vesical artery 160 to the site of the stenosis (not shown). For reference, the left common iliac artery and abdominal aorta are indicated at 135 and 140, respectively. In alternate embodiments, stent delivery portion 130 may be maneuvered through internal iliac artery 105 into one of the superior vesical artery 164, the umbilical artery 162, the internal pudendal artery 145, the middle rectal artery 165, or other pelvic artery to the site of a stenosis (not shown) for placement of a stent. For an application as shown in FIG. 1, the length of guide catheter portion 125 of system 100 may be in the range of 30 centimeters to 130 centimeters, and in one embodiment the length of guide catheter portion 125 may be approximately 50 centimeters.

A stent for use in embodiments of the present invention may be balloon expandable or self-expanding and may be made from any suitable medically implantable material, such as, but not limited to, stainless steel, nitinol, tantalum, ceramic, nickel, titanium, aluminum, polymeric materials, cobalt alloys, magnesium, platinum iridium, titanium ASTM F63-83 Grade 1, niobium, high carat gold K 19-22, and combinations thereof. Stents and structures for stents suitable for use in embodiments of the present invention are disclosed in U.S. Pat. No. 4,733,665 to Palmaz, U.S. Pat. No. 4,800,882 to Gianturco, U.S. Pat. No. 4,886,062 to Wiktor, U.S. Pat. No. 5,133,732 to Wiktor, U.S. Pat. No. 5,292,331 to Boneau, U.S. Pat. No. 5,421,955 to Lau, U.S. Pat. No. 5,935,162 to Dang, U.S. Pat. No. 6,090,127 to Globerman, and U.S. Pat. No. 6,730,116 to Wolinsky et al., each of which is incorporated by reference herein in its entirety. As well, any suitable guide catheter and/or stent delivery catheter may be utilized in embodiments of the present invention, with or without a guidewire, as would be apparent to one of ordinary skill in the art.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A method of diagnosing lower urinary tract symptoms correctable by arterial stenting, the method comprising:
identifying a stenosis in a pelvic artery that is restricting blood flow to an artery that supplies blood to one of the urinary bladder, the urethra, a nerve innervating the urinary bladder, and a nerve innervating the urethra.

2. The method of claim 1, wherein the step of identifying a stenosis in a pelvic artery includes performing a pelvic angiogram.

3. The method of claim 1, wherein the step of identifying a stenosis in a pelvic artery includes performing a diagnostic procedure selected from the group consisting of a CT scan, a MRI, CT-angiography, MR-angiography, doppler sonography, color duplex sonography, and nuclear imaging.

4. A method of treating lower urinary tract symptoms caused by atherosclerosis in a pelvic artery that supplies blood to one of the urinary bladder, the urethra, a nerve innervating the urinary bladder, and a nerve innervating the urethra, the method comprising:
tracking a delivery catheter having a stent to a stenosis within the artery; and
restoring blood flow through the artery by expanding the stent within the stenosis.

5. The method of claim 4, wherein the pelvic artery is one of the internal iliac artery, external iliac artery, and common iliac artery.

6. The method of claim 4, wherein the pelvic artery is one of the superior vesical artery, the umbilical artery, the vaginal artery, the obturator artery, and the inferior gluteal artery.

7. The method of claim 4, wherein the pelvic artery is one of the internal pudendal artery, the inferior vesical artery, and the middle rectal artery.

8. The method of claim 4, wherein the pelvic artery is one of a prostatic artery, the artery to the urethral bulb, and the urethral artery.

9. The method of claim 4, wherein pelvic artery supplies blood to one of the vesical nerve plexus, the pudendal nerve, and the cavernous nerve plexuses.

10. The method of claim 4, wherein the stent used in the step of restoring blood flow through the artery is balloon expandable.

11. The method of claim 4, wherein the stent used in the step of restoring blood flow through the artery is self-expanding.

12. The method of claim 4, wherein the stent used in the step of restoring blood flow through the pelvic artery is a drug-eluting stent.

13. A method of treating lower urinary tract symptoms caused by atherosclerosis in a pelvic artery, the method comprising:
tracking a revascularization tool to a stenosis within the pelvic artery; and
performing a revascularization procedure with the revascularization tool within the pelvic artery to substantially eliminate the stenosis and restore flow through the pelvic artery.

14. The method of claim 13, wherein the revascularization procedure includes rotoblading.

15. The method of claim 13, wherein the revascularization tool includes a vibrating portion for breaking-up the stenosis via mechanical vibration.

16. The method of claim 13, wherein the revascularization tool includes using saline or venturi jet devices for softening the stenosis for removal.

17. The method of claim 13, wherein the revascularization procedure is an intravascular brachytherapy and the revascularization tool includes a radiation source that is implantable within the stenosis.

18. The method of claim 13, wherein the revascularization tool includes a clot retrieval mechanism.

19. The method of claim 13, wherein the revascularization tool utilizes ultrasound for breaking-up the stenosis.

20. The method of claim 13, wherein the revascularization procedure is angioplasty and the revascularization tool includes a balloon catheter.
